(19) United States
(12) Patent Application Publication
(10) Pub. No.: US 2020/0146984 A1
(43) Pub. Date: May 14, 2020

(54) COMBINATION OF CANNABINOIDS AND AT LEAST ONE ADDITIONAL INGREDIENT FOR THE ENHANCEMENT OF THERAPEUTIC POTENCY

(71) Applicant: PANAXIA PHARMACEUTICAL INDUSTRIES LTD., Lod (IL)

(72) Inventors: Dadi David SEGAL, Tel-Aviv (IL); Or SEGAL, Holon (IL); Eran GOLDBERG, Ramat Gan (IL)

(73) Assignee: PANAXIA PHARMACEUTICAL INDUSTRIES LTD., Lod (IL)

(21) Appl. No.: 16/625,837

(22) PCT Filed: Jun. 27, 2018
(86) PCT No.: PCT/IL2018/050704

§ 371 Date: Dec. 23, 2019

Related U.S. Application Data
(60) Provisional application No. 62/525,452, filed on Jun. 27, 2017.
(60) Provisional application No. 62/525,456, filed on Jun. 27, 2017.
(60) Provisional application No. 62/525,462, filed on Jun. 27, 2017.

(51) Int. Cl. A61K 9/06; A61K 36/185; A61K 47/44; A61K 47/10; A61K 47/36; A61K 36/53; A61K 36/45; A61K 36/537; A61K 36/28; A61K 31/352

(52) U.S. Cl. CPC A61K 9/06; A61K 36/185; A61K 47/44; A61K 47/10; A61K 31/352; A61K 36/53; A61K 36/45; A61K 36/537; A61K 36/28; A61K 47/36

(57) ABSTRACT

Disclosed is a composition comprising a cannabinoid and at least one of an essential oil, an herbal extract, a sleep aid-compound, or any combination thereof. Further disclosed is a method for treating a condition modulated by the activation of cannabinoid receptors CB1, CB2, or both, wherein the method comprising administering the composition of the invention. Further disclosed is a method for preparing a cannabis gel.

### FIELD OF THE INVENTION

This invention is directed to a composition comprising a combination of at least one cannabinoid and at least one of essential oil, herbal extract or sleep aid, wherein the at least one essential oil herbal extract or sleep aid, enhances the therapeutic potency of the composition.

### BACKGROUND OF THE INVENTION

Cannabis legalisation is spreading throughout the world, especially for various medicinal treatments. Therefore, the use of cannabis is growing world-wide. However, the therapeutic potency of known cannabis products is limited, especially when administered orally.

Accordingly, there is a need for cannabis products, including oral ones, having an enhanced pharmaceutical potency.

### SUMMARY OF THE INVENTION

Embodiments of the invention are directed to a cannabis gel comprising:

cannabis extract in an amount of about 0.1-3% w/w;
an emulsifying agent in an amount that is about 3-20 times higher than the amount of the extract;
water in an amount that is about 5-40 times higher than the amount of the extract;
a gelling agent in an amount of about 0.1-5% w/w;
a non-aqueous diluent in an amount of about 30-80% w/w; and
flavoring agents, any pharmaceutical carrier or additive, or any combination thereof, in an amount that is up to about 5%.

According to some embodiments, the amount of all of the additives, including carriers, flavorings, etc. is up to about 5%. According to some embodiments, the amount of each additive or type of additive, such as a flavoring agent is up to about 5%.

According to some embodiments, the cannabis gel comprises:

17% distilled extract;

9.36% emulsifying agent;

29.25% water;

0.2% flavoring agent;

0.5% gelling agent; and

59.52% non-aqueous diluent.

According to some embodiments, the gelling agent is agar, acacia, alginic acid, bentonite, carbopols, carboxymethyl cellulose, ethylcellulose, gelatin, hydroxyethyl cellulose, hydroxypropyl cellulose, magnesium aluminum silicate (Veegum®), methylcellulose, poloxamers (Pluronics®), polyvinyl alcohol, sodium alginate, tragacanth, or xanthan gum.

According to some embodiments, the non-aqueous diluent is glycerin, propylene glycol, polyethylene glycol or polyol. According to some embodiments, the emulsifying agent is polyoxyl castor oil.

According to some embodiments the cannabis gel comprises:

1.17% distilled extract;

9.36% polyoxyl castor oil;

29.25% water;

0.2% menthol;

0.5% agar; and

59.52% glycerin.

Some embodiments of the invention are directed to a composition comprising the cannabis gel of the invention, wherein the cannabis gel comprises cannabinoid and wherein the composition further comprises at least one of:

an essential oil;

an herbal extract; and

a sleep aid-compound.

According to some embodiments, the cannabinoid comprises tetrahydrocannabinol (THC), cannabidiol (CBD), cannabinol (CBN), or any combination thereof. According to some embodiments, the essential oil is obtained from cinnamomum leaf, salvia sclarea, canarium luzonicum, Origanum vulgare, or any combination thereof. According to some embodiments, the herbal extract is an extract of silybum marianum, cranberry, clove, or any combination thereof. According to some embodiments, the sleep aid compound is doxylamine, diphenhydramine, melatonin, or any combination thereof.

According to some embodiments, the comprising comprises between about 3-200 mg cannabinoids per dosage unit. According to some embodiments, the composition comprises between about 0.1-50 mg sleep-aid compound. According to some embodiments, the amount of each of the essential oil or the herbal extract is up to about 1% w/w. According to some embodiments, the amount of each of the essential oil or the herbal extract is up to about 5% w/w. According to some embodiments, the amount of each of the essential oil or the herbal extract is up to about 10% w/w. According to some embodiments, the amount of each of the essential oil or the herbal extract is up to about 15% w/w. According to some embodiments, the amount of each of the essential oil or the herbal extract is up to about 20% w/w. According to some embodiments, the composition is in the form of a gel.

Further embodiments of the invention are directed to a method for treating a condition modulated by the activation of cannabinoid receptors CB1, CB2, or both, wherein the method comprises administering the composition of the invention or the gel of the invention.

According to some embodiments, the condition is a sleep disorder and wherein the composition comprises a sleep-aid compound. According to some embodiments, the sleep disorder is insomnia.

Further embodiments of the invention are directed to a method for preparing a cannabis gel, wherein the method comprises:
mixing a cannabis extract with an emulsifying agent in a gel preparation vessel;
adding water to the gel preparation vessel and mixing;
optionally adding a flavoring agent to the gel preparation vessel and mixing;
adding a gelling agent and a non-aqueous diluent to the gel preparation vessel, mixing and heating to between about 85-95° C. cooling to between about 40-55° C. to obtain a homogeneous mixture;
optionally transferring the homogeneous mixture into storage vessels;
storing the homogeneous mixture in the storage vessels or in the gel preparation vessel for at least about 8 hours, and, during storage, allowing the homogeneous mixture to cool to room temperature, thereby providing the cannabis gel.

Further embodiments of the invention are directed to a method for preparing a cannabis gel, wherein the method comprises:
mixing a cannabis extract with an emulsifying agent in a gel preparation vessel;
adding water to the gel preparation vessel and mixing;
optionally adding a flavoring agent to the gel preparation vessel and mixing;
adding a gelling agent and one or more of the group consisting of glycerin, propylene glycol or any sugar alcohol to the gel preparation vessel;
mixing so as to obtain a homogeneous mixture;
optionally transferring the homogeneous mixture into storage vessels;
storing the homogeneous mixture in the storage vessels or in the gel preparation vessel for at least about 8, 10 or 12 hours, and, during storage, allowing the homogeneous mixture to cool to room temperature, thereby providing the cannabis gel.

According to some embodiments, the cannabis extract and the emulsifying agent are mixed together and are heated for about 2.5, 5, 7.5, 10, 12.5, 15, 19.5 or 20 minutes at a temperature of about 70° C., 75° C., 80° C., 85° C. or 90° C.

According to some embodiments, the emulsifying agent is polyoxyl castor oil. According to some embodiments, the gelling agent is agar, acacia, alginic acid, bentonite, carbopols, carboxymethyl cellulose, ethylcellulose, gelatin, hydroxyethyl cellulose, hydroxypropyl cellulose, magnesium aluminum silicate (Veegum®), methylcellulose, poloxamers (Pluronics®), polyvinyl alcohol, sodium alginate, tragacanth, or xanthan gum.

According to some embodiments, the flavoring agent is selected from the group consisting of menthol, camphor, saccharin, sucralose, sugar, sucrose, sorbitol, sucralose, saccharin sodium, sodium cyclamate, aspartame, neotame, acesulfame potassium, stevioside, sodium chloride, D-limonene, citric acid and any combination thereof.

Further embodiments of the invention are directed to a composition comprising a cannabinoid and at least one of:

an essential oil;

an herbal extract; and

a sleep aid-compound.

According to some embodiments, the cannabinoid comprises tetrahydrocannabinol (THC), cannabidiol (CBD), cannabinol (CBN), or any combination thereof. According to some embodiments, the essential oil is obtained from cinnamomum leaf, salvia sclarea, canarium luzonicum, Origanum vulgare, or any combination thereof. According to some embodiments, the herbal extract is an extract of silybum marianum, cranberry, clove, or any combination thereof. According to some embodiments, the sleep aid compound is doxylamine, diphenhydramine, melatonin, or any combination thereof.

According to some embodiments, the composition comprises between about 3-200 mg cannabinoids per dosage unit. According to some embodiments, the composition comprises between about 5-150 mg cannabinoids per dosage unit. According to some embodiments, the composition comprises between about 10-100 mg cannabinoids per dosage unit. According to some embodiments, the composition comprises between about 0.1-50 mg sleep-aid compound. According to some embodiments, the composition comprises between about 0.5-30 mg sleep-aid compound. According to some embodiments, the composition comprises between about 1-10 mg sleep-aid compound. According to some embodiments, the composition comprises between about 5-50 mg sleep-aid compound. According to some embodiments, the composition comprises between about 5-10 mg sleep-aid compound. According to some embodiments, the essential oil and the herbal extract is between about 2-90% w/w. According to some embodiments, the amount of the essential oil is between about is between about 2-90% w/w. According to some embodiments, the amount of the essential oil is between about is between about 2-10% w/w. According to some embodiments, the amount of the herbal extract is between about is between about 2-20% w/w. According to some embodiments, the amount of the herbal extract is between about is between about 2-30% w/w. According to some embodiments, the amount of the herbal extract is between about is between about 2-40% w/w. According to some embodiments, the amount of the herbal extract is between about is between about 30-50% w/w. According to some embodiments, the amount of the herbal extract is between about is between about 50-90% w/w.

According to some embodiments, the amount of the essential oil is between about is between about 2-10% w/w According to some embodiments, the amount of the essential oil is between about is between about 2-20% w/w. According to some embodiments, the amount of the essential oil is between about is between about 2-30% w/w. According to some embodiments, the amount of the essential oil is between about is between about 2-40% w/w. According to some embodiments, the amount of the essential oil is between about is between about 30-50% w/w. According to some embodiments, the amount of the essential oil is between about is between about 50-90% w/w.

According to some embodiments, the composition is in the form of a gel.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Throughout this description, the term “about” is intended to cover ±10% of the specifically disclosed value.

Embodiments of the invention are directed to a composition comprising at least one cannabinoid, e.g., tetrahydrocannabinol (THC) or cannabidiol (CBD), and at least one small molecule selected from the essential oils of cinnamomum leaf, salvia sclarea, canarium luzonicum, Origanum vulgare, or any combination thereof, wherein the composition exhibits enhancement in therapeutic potency and increased duration of action of THC and/or CBD in comparison to composition to which the essential oil was not added.

Further embodiments of the invention are directed to methods of treating one or more disease(s) or disorder(s) modulated by the activation of the Cannabinoid CB1 and/or CB2 receptors, such as, but not limited to, PTSD, chronic pain, cancer, painful peripheral neuropathy, intractable nausea/vomiting, HIV/AIDS, epilepsy, multiple sclerosis, spinal cord damage, arthritis, glaucoma, anorexia, Crohn's disease, Parkinson's disease, ALS, and spasmodic torticollis, by administering a therapeutically effective amount of a composition comprising at least one cannabinoid, for example, tetrahydrocannabinol (THC) or cannabidiol (CBD), and at least one small molecule selected from the essential oils of cinnamomum leaf, salvia sclarea, canarium luzonicum, Origanum vulgare, or any combination thereof, to a subject in need thereof.

In addition, embodiments of the invention are directed to a pharmaceutical composition comprising one or more cannabinoids, such as, for example, THC and CBD, and at least one small molecule selected from the herbal extracts of silybum marianum, cranberry, clove, or any combination thereof, wherein the composition exhibits an enhancement in therapeutic potency and increased duration of action of the cannabinoids, in comparison to compositions to which the herbal extract was not added.

As used herein, the term “cannabis extract” refers to any extract that can be obtained by any suitable technique for extracting cannabis resin from a cannabis plant.

The extracts may be produced by cold extraction techniques using a variety of different extraction solvents including water, fatty solvents (such as olive oil), and alcoholic solvents (e.g. 70% ethanol). Cold extraction techniques are typically applied to softer parts of the plant, such as, leaves and flowers, or in cases wherein the desired active components of the plant are heat labile. Alternatively, the solvents may be used to produce extracts of the desired plants by a hot extraction technique, wherein said solvents are heated to a high temperature, the precise value of said temperature being dependent on the properties of the chosen solvent and maintained at that temperature throughout the extraction process. Hot extraction techniques are more commonly applied to the harder, tougher parts of the plant, such as bark, woody branches and larger roots. In some cases, sequential extractions can be performed in more than one solvent, and at different temperatures. The plant extract may be used in a concentrated form. Alternatively, the extract may be diluted as appropriate to its intended use.

Procedures for producing plant extracts (including hot extraction, cold extraction and other techniques) are described in publications including “Medicinal plants: a field guide to the medicinal plants of the Land of Israel (in Hebrew), author: N. Krispil, Har Gilo, Israel, 1986” and “Making plant medicine, author: R. Cech, pub. by Horizon Herbs, 2000”.

As a non-limiting example, the extract can be obtained by simply shaking or scraping resin off a Cannabis plant.

As another non-limiting example, butane or carbon dioxide can be used as a solvent for extraction.

As another non-limiting example, the Cannabis plant may be dried and ground into fine material. The ground material may then be mixed with a food-grade alcohol (such as at a ratio of 1lb of ground material to 1.5 gallons of alcohol), so that the ground material becomes fully immersed in the alcohol. The mixture may then be placed in a covered container and heated for 3 hours at 190° F., while refluxing the alcohol so as to distill the oils from the ground material. After allowing the mixture to cool, it may be passed through a strainer to separate liquid from solid plant residue. The liquid may then be separated such as by pouring through a filter. The filtered liquid contains alcohol and Cannabis extract. In order to remove the alcohol, the filtered liquid may be subjected to an evaporative process, such as heating, leaving the Cannabis extract behind.

As another non-limiting example, the Cannabis plant may be dried and ground into fine material, and then immersed in alcohol. The mixture may then be filtered, separating the liquid from the plant material. The alcoholic liquid containing diluted cannabinoids may then be partially, evaporated to reach a high concentration of cannabinoids. The concentrated alcoholic extract may subsequently be winterized to remove waxes by incubation in sub-zero temperature, sedimentation of the waxes and cold filtration of the mixture. The remaining alcohol may then be evaporated to obtain an extract in the form of a resin. The extract may then be decarboxylated by placing in an oven at ≥100° C. for at least 1 hour. An extract with 40-90% cannabinoid content is thereby typically obtained.

Further embodiments of the invention are directed to methods of treating one or more disease(s) or disorder(s) modulated by the activation of the Cannabinoid CB1 and/or CB2 receptors, by administering a therapeutically effective amount of a pharmaceutical composition comprising one or more cannabinoids, such as, for example, THC and CBD, and at least one small molecule selected from the herbal extracts of silybum marianum, cranberry, clove, or any combination thereof, to a subject in need thereof.

According to some embodiments, the composition of the invention comprises both an essential oil and an herbal extract together with at least one cannabinoid. Accordingly, methods of the invention include administering a composition comprising at least one cannabinoid, at least one essential oil and at least one herbal extract.

Embodiments of the invention are directed to a pharmaceutical composition comprising one or more cannabinoid(s), such as, for example, cannabinol (CBN) and/or Cannabidiol (CBD), and at least one sleep-aid compound, such as, for example, doxylamine, diphenhydramine, melatonin, or any combination thereof, wherein the composition exhibits enhanced sedative potency, in comparison to composition to which the sleep aid compound or the cannabinoid has not been added. According to some embodiments, the composition of the invention comprising at least one cannabinoid and at least one sleep-aid compound is used for treating sleep disorders, such as for example, insomnia.

According to some embodiments, the composition of the invention comprises an essential oil and an herbal extract together with at least one cannabinoid, at least one sleep aid compound. Accordingly, methods of the invention include administering a composition comprising at least one cannabinoid, at least one essential oil, at least one herbal extract, at least one sleep aid compound.

According to some embodiments, the composition includes between about 3-200 mg cannabinoids per single dose. According to some embodiments, the composition is a combination of at least two cannabinoids, wherein the combined amount of the cannabinoids in the composition is between about 3-200 mg per dosage unit. According to some embodiments, the cannabinoids are in the form of an extract.

According to some embodiments, the composition comprises 3, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 mg cannabinoid per dosage unit, e.g., THC, CBD and/or CBN.

As mentioned above, the composition may further include a sleep aid compound. According to some embodiments, the composition comprises between about 0.1-50 mg of a sleep aid compound per dosage unit. According to some embodiments, the composition comprises about 0.1, 0.3, 0.5, 0.75, 1, 3, 5, 7.5, 10, 15, 20, 25, 30, 35, 40, 45 or 50 mg of a sleep aid component per dosage unit. According to some embodiments, the sleep aid component is melatonin.

According to some embodiments the composition comprises about 5 mg CBD, about 5 mg CBN, and about 3 mg melatonin per dosage unit.

According to some embodiments, the composition comprises about 10 mg CBD, about 10 mg CBN and about 5 mg melatonin per dosage unit.

According to some embodiments, the dosage form is an immediate release form. According to some embodiments, the dosage form is a controlled release dosage form, e.g., a slow release form. According to some embodiments, the dosage form is in the form of a gel.

According to some embodiments, an immediate release form may be administered together with a slow release form of the composition of the invention. According to some embodiments the slow release composition comprises about 5 mg CBD, about 5 mg CBN and about 3 mg melatonin per dosage unit. According to some embodiments, the immediate release composition comprises about 10 mg CBD, about 10 mg CBN and about 5 mg melatonin per dosage unit. According to some embodiments, the above slow release composition is administered together with the above immediate release composition in order to treat the patient both immediately and over a prolonged period of time.

According to some embodiments, the composition of the invention comprises 20 g Cannabis extract (50% cannabinoids), 50 g essential oil and 50 g herbal extract.

According to some embodiments, the composition of the invention comprises 20 g Cannabis extract (50% THC), 50 g Origanum vulgare essential oil and 50 g cranberry extract.

According to some embodiments, the composition of the invention comprises 20 g Cannabis extract (50% THC), 50 g salvia sclarea essential oil and 50 g silybum marianum extract.

Embodiments of the invention are directed to a cannabis gel comprising:

Cannabis Extract (about 0.1, 0.2, 0.3, 0.4, 0.5, 0.7, 0.9, 1.1, 1.3, 1.5, 2, 2.1, 2.3, 2.5, 2.7, 2.9 or 3% w/w);

emulsifying agent (in an amount which is 3-20 times the % of the extract);

water (in an amount which is 5-40 times the % of the extract);

gelling agent (about 0.1, 0.2, 0.3, 0.4, 0.5, 0.7, 0.9, 1, 1.5, 2, 2.5, 3, 3.5, 4, 4.5, 4.7, or 5% w/w);

Non-aqueous diluent (such as without limitation glycerin, propylene glycol, polyethylene glycol, polyol) (about 30, 40, 50, 60, 70, or 80% w/w); and

Flavoring agents, such as sweeteners (up to 5%). According to some embodiments, the flavoring agent is a sweetener.

Embodiments of the invention are directed to a method for preparing a cannabis gel, wherein the method comprises:
mixing a cannabis extract with an emulsifying agent in a gel preparation vessel;
adding water to the gel preparation vessel and mixing;
optionally adding a flavoring agent to the gel preparation vessel and mixing;
adding gelling agent and a non-aqueous diluent;
to the gel preparation vessel, mixing and heating to between about 85-95° C.;
cooling to between about 40-55° C. to obtain a homogeneous mixture;
optionally transferring the homogeneous mixture into storage vessels;
storing the homogeneous mixture in the storage vessels or in the gel preparation vessel for at least 8, 10 or 12 hours, and, during storage allowing the homogeneous mixture to cool to room temperature, thereby providing the cannabis gel.

In some embodiments of the invention, the gelling agent is without limitation one or more of the group consisting of agar, acacia, alginic acid, bentonite, Carbopols (now known as carbomers), carboxymethyl cellulose, ethylcellulose, gelatin, hydroxyethyl cellulose, hydroxypropyl cellulose, magnesium aluminum silicate (Veegum), methylcellulose, poloxamers (Pluronics), polyvinyl alcohol, sodium alginate, tragacanth, and xanthan gum.

In some embodiments of the invention, the flavoring agent is one or more agent selected from the group consisting of menthol, camphor, saccharin, sucralose, sugar, sucrose, sorbitol, sucralose, saccharin sodium, sodium cyclamate, aspartame, neotame, acesulfame potassium, stevioside, sodium chloride, D-limonene, citric acid and any combination thereof.

In some embodiments if the gelling agent does not require a step of heating the method for preparing a gel may be as follows:

mixing a cannabis extract with an emulsifying agent in a gel preparation vessel;

adding water to the gel preparation vessel and mixing;

optionally adding a flavoring agent to the gel preparation vessel and mixing;

adding gelling agent and a non-aqueous diluent;

to the gel preparation vessel, mixing so as to obtain a homogeneous mixture;

optionally transferring the homogeneous mixture into storage vessels;

storing the homogeneous mixture in the storage vessels or in the gel preparation vessel for at least 8, 10 or 12 hours, and, during storage allowing the homogeneous mixture to cool to room temperature, thereby providing the cannabis gel.

According to some embodiments, the cannabis extract and the emulsifying agent are mixed together and heated for about 2.5, 5, 7.5, 10, 12.5, 15, 17.5 or 20 minutes, e.g., in an oil bath at a temperature of about 70° C., 75° C., 80° C., 85° C. or 90° C. According to some embodiments, the emulsifying agent is polyoxyl castor oil, also known as Kolliphor RH40.

According to some embodiments, the gelling agent is agar.

In some embodiments of the invention, the flavoring agent is one or more agent selected from the group consisting of menthol, camphor, saccharin, sucralose, sugar, sucrose, sorbitol, sucralose, saccharin sodium, sodium cyclamate, aspartame, neotame, acesulfame potassium, stevioside, sodium chloride, D-limonene, citric acid and any combination thereof.

Various aspects of the invention are described in greater detail in the following Examples, which represent embodiments of this invention, and are by no means to be interpreted as limiting the scope of this invention.

### EXAMPLES

### Example 1—Preparation of Cannabis Extract

Cannabis plant material is ground to a fine powder and cooled to 0° C. Then it is fully covered with cold (0° C.) ethanol for 30 minutes. The mixture is then filtered, separating the liquid from the plant material. The ethanoic liquid containing diluted cannabinoids is then partially evaporated to reach a high concentration of cannabinoids. The concentrated ethanoic extract is winterized to remove waxes by incubation in sub-zero temperature, sedimentation of the waxes and cold filtration of the mixture. The remaining ethanol is then evaporated and an extract in the form of a resin is retained. The extract is then decarboxylated by placing in a 100° C. oven for 4 hours. An extract with 40-90% Cannabinoid content is obtained.

### Example 2—Preparation of Cannabinoid, Herbal Extracts and Essential Oils Mix

Mix 1: 20 g Cannabis extract (50% THC), 50 g Origanum vulgare essential oil and 50 g cranberry extract were homogenized using an overhead stirrer in a container placed in a hot water bath.

Mix 2: 20 g Cannabis extract (50% THC), 50 g salvia sclarea essential oil and 50 g silybum marianum extract were homogenized using an overhead stirrer in a container placed in a hot water bath.

### Example 3—Subjective Efficacy Evaluation of Cannabinoid, Herbal Extracts, and Essential Oils Mix (Prophetic Example)

Subjective efficacy and duration testing will be performed in order to evaluate the onset of action, duration of effect and the subjective therapeutic efficacy of cannabinoid extract alone compared to formulations of the invention which comprise at least one cannabinoid with specific combinations of herbal extracts and/or essential oils. Formulations of cannabis extract with essential oil and/or herbal essence will be given to subjects, while other subjects will be given a control formulation of cannabis extract alone. Every five minutes during one hour, starting immediately before administering, the subjects will report their subjective evaluation of the cannabis effect on a scale of 0-4, according to the following definitions: 0—without any cannabis effect, 1—slight cannabis effect, 2—moderate cannabis effect, 3—intense cannabis effect, 4—very intense cannabis effect. The subject will continue to report their subjective score as above until the effect reaches 0. This measurement will be used to assess the increase in the duration of the effect.

### Example 4—Preparation of Cannabinoid Gel

The cannabis extract is readily soluble in oils such as canola oil, which may be used as a carrier oil for oral administration of the cannabis extract. However, the use of canola oil as a carrier presents some disadvantages, such as poor taste of the final product (difficult to mask the bitter taste of cannabis), poor bioavailability (low solubility in the gastrointestinal tract) and high caloric value.

In the experiments detailed below, the oil-in-water solubilizer was used together with the emulsifying agent, such as, polyoxyl castor oil also known as Kolliphor RH40, thereby replacing the canola oil. Glycerin was also used as the major component of these formulations. Small quantities of water (20 to 25%) were added in order to solubilize the Kolliphor and to prepare initial concentrated emulsions. Agar, a gelling agent obtained from algae (with melting point 85 to 95° C. and setting point 32 to 45° C.), was also tested to enhance the mechanical stability of the formulation.

### Ingredients:

1.17% distilled extract
9.36% Kolliphor RH40 (calculated as being eight times the percentage of the extract)
29.25% Water (calculated as being 25 times the percentage of the extract)

### 0.2% Menthol

### 0.5% Agar

### 59.52% Glycerin

A distilled extract with 77.8% THC was used. Since 1.1 gr gel is required in each single dose container, and since the preparation is intended to comprise 10 mg THC in each single dose (10/0.778=12.85 mg extract), the extract concentration was calculated as 12.85/1100=1.17%. The percentage of Kolliphor (% extract X 8) and water (X 25) were chosen to obtain a stable emulsion.

100 gr gel were prepared in a 200 mL beaker. All instruments were disinfected with ethanol and further, sterile water was used for irrigation.

An impeller was used for homogenization throughout the process, except when mixing the extract and the Kolliphor RH40, which were homogenized using a spatula.

### Process:

1.175 gr extract and 9.33 gr Kolliphor RH40 were weighed together in a 200 mL beaker and heated for five minutes in an oil bath at 80° C. A spatula was used to homogenize. The beaker was removed from oil bath and 29.26 gr water were added. An impeller and a spatula were used to homogenize the mixture until a clear yellow emulsion was obtained.

0.206 gr menthol were added to the emulsion, which was further homogenized using an impeller until the complete dissolution of the menthol in the emulsion.

0.504 gr agar were then dispersed in the solution and 59.51 gr glycerin were added. An impeller was applied during heating to 90° C. for one minute for the dissolution of Agar. Stirring continued while cooling down until reaching 60° C.

After cooling down to 50° C. the liquid was poured into a sterile cup for analysis. The gel started to set after about one hour and was left overnight for complete setting of the gel.

1. Cannabis gel comprising:
cannabis extract in an amount of 0.1-3% w/w;
an emulsifying agent in an amount that is 3-20 times higher than the amount of the extract;
water in an amount that is 5-40 times higher than the amount of the extract;
a gelling agent in an amount of 0.1-5% w/w;
a non-aqueous diluent in an amount of 30-80% w/w; and
flavoring, any pharmaceutical carrier or additive, or any combination thereof, in an amount that is up to 5%.

2. The cannabis gel of claim 1 comprising:
17% distilled extract;
9.36% emulsifying agent;
29.25% water;
0.2% flavoring agent;
0.5% gelling agent; and
59.52% non-aqueous diluent.

3. The cannabis gel of any one of claim 1 or 2, wherein the gelling agent is agar, acacia, alginic acid, bentonite, carbopols, carboxymethyl cellulose, ethylcellulose, gelatin, hydroxyethyl cellulose, hydroxypropyl cellulose, magnesium aluminum silicate (Veegum®), methylcellulose, poloxamers (Pluronics®), polyvinyl alcohol, sodium alginate, tragacanth, or xanthan gum.

4. The cannabis gel of any one of claims 1-3, wherein the non-aqueous diluent is glycerin, propylene glycol, polyethylene glycol or polyol.

5. The cannabis gel of any one of claims 1-4, wherein the emulsifying agent is polyoxyl castor oil.

6. The cannabis gel of any one of claims 1-5 comprising:
1.17% distilled extract;
9.36% polyoxyl castor oil;
29.25% water;
0.2% menthol;
0.5% agar; and
59.52% glycerin.

7. A composition comprising the cannabis gel of any one of claims 1-6, wherein the cannabis gel comprises cannabinoid and wherein the composition further comprises at least one of:
an essential oil;
an herbal extract; and
a sleep aid-compound.

8. The composition according to claim 7, wherein the cannabinoid comprises tetrahydrocannabinol (THC), cannabidiol (CBD), cannabinol (CBN), or any combination thereof.

9. The composition according to claim 7, wherein the essential oil is obtained from cinnamomum leaf, salvia sclarea, canarium luzonicum, Origanum vulgare, or any combination thereof.

10. The composition according to claim 7, wherein the herbal extract is an extract of Silybum marianum, cranberry, clove, or any combination thereof.

11. The composition according to claim 7, wherein the sleep aid compound is doxylamine, diphenhydramine, melatonin, or any combination thereof.

12. The composition according to claim 7, comprising between 3-200 mg cannabinoids per dosage unit.

13. The composition according to claim 7, comprising between 0.1-50 mg sleep-aid compound.

14. The composition of claim 7, wherein the amount of each of the essential oil or the herbal extract is up to 20% w/w.

15. The composition according to claim 7, wherein the composition is in the form of a gel.

16. Method for treating a condition modulated by the activation of cannabinoid receptors CB1, CB2, or both, said method comprising administering the composition according to any one of claims 7-16 or the gel according to any one of claims 1-6.

17. The method according to claim 16, wherein the condition is a sleep disorder and wherein the composition comprises a sleep-aid compound.

18. The method according to claim 17, wherein the sleep disorder is insomnia.

19. Method for preparing a cannabis gel, said method comprising:
mixing a cannabis extract with an emulsifying agent in a gel preparation vessel;
adding water to the gel preparation vessel and mixing;
optionally adding a flavoring agent to the gel preparation vessel and mixing;
adding a gelling agent and a non-aqueous diluent to the gel preparation vessel, mixing and heating to between about 85-95° C. cooling to between about 40-55° C. to obtain a homogeneous mixture;
optionally transferring the homogeneous mixture into storage vessels;
storing the homogeneous mixture in the storage vessels or in the gel preparation vessel for at least 8 hours, and, during storage, allowing the homogeneous mixture to cool to room temperature, thereby providing the cannabis gel.

20. Method for preparing a cannabis gel, said method comprising:
mixing a cannabis extract with an emulsifying agent in a gel preparation vessel;
adding water to the gel preparation vessel and mixing;
optionally adding a flavoring agent to the gel preparation vessel and mixing;
adding a gelling agent and one or more of the group consisting of glycerin, propylene glycol or any sugar alcohol to the gel preparation vessel;
mixing so as to obtain a homogeneous mixture;
optionally transferring the homogeneous mixture into storage vessels;
storing the homogeneous mixture in the storage vessels or in the gel preparation vessel for at least 8, 10 or 12 hours, and, during storage, allowing the homogeneous mixture to cool to room temperature, thereby providing the cannabis gel.

21. The method according to any one of claims 19 and 20, wherein the cannabis extract and the emulsifying agent are mixed together and are heated for about 2.5, 5, 7.5, 10, 12.5, 15, 19.5 or 20 minutes at a temperature of about 70° C., 75° C., 80° C., 85° C. or 90° C.

22. The method according to any one of claims 19 and 20, wherein the emulsifying agent is polyoxyl castor oil.

23. The method according to any one of claims 19 and 20, wherein the gelling agent is agar, acacia, alginic acid, bentonite, carbopols, carboxymethyl cellulose, ethylcellulose, gelatin, hydroxyethyl cellulose, hydroxypropyl cellulose, magnesium aluminum silicate (Veegum®), methylcellulose, poloxamers (Pluronics®), polyvinyl alcohol, sodium alginate, tragacanth, or xanthan gum.

24. The method of any one of claims 19 and 20, wherein the flavoring agent is selected from the group consisting of menthol, camphor, saccharin, sucralose, sugar, sucrose, sorbitol, sucralose, saccharin sodium, sodium cyclamate, aspartame, neotame, acesulfame potassium, stevioside, sodium chloride, D-limonene, citric acid and any combination thereof.

25. Composition comprising a cannabinoid and at least one of:
an essential oil;
an herbal extract; and
a sleep aid-compound.

26. The composition according to claim 25, wherein the cannabinoid comprises tetrahydrocannabinol (THC), cannabidiol (CBD), cannabinol (CBN), or any combination thereof.

27. The composition according to claim 25, wherein the essential oil is obtained from cinnamomum leaf, salvia sclarea, canarium luzonicum, Origanum vulgare, or any combination thereof.

28. The composition according to claim 25, wherein the herbal extract is an extract of silybum marianum, cranberry, clove, or any combination thereof.

29. The composition according to claim 25, wherein the sleep aid compound is doxylamine, diphenhydramine, melatonin, or any combination thereof.

30. The composition according to claim 25, comprising between 3-200 mg cannabinoids per dosage unit.

31. The composition according to claim 25, comprising between 0.1-50 mg sleep-aid compound.

32. The composition of claim 25, wherein the amount of each of the essential oil or the herbal extract is between 2-90% w/w.

33. The composition according to claim 25, wherein the composition is in the form of a gel.
